(19) United States
(12) Patent Application Publication
(10) Pub. No.: US 2021/0142173 A1
(43) Pub. Date: May 13, 2021

(54) NETWORK-BASED DEEP LEARNING TECHNOLOGY FOR TARGET IDENTIFICATION AND DRUG REPURPOSING

(71) Applicant: THE CLEVELAND CLINIC FOUNDATION, Cleveland, OH (US)

(72) Inventor: Feixiong Cheng, Cleveland, OH (US)

(21) Appl. No.: 17/096,712

(22) Filed: Nov. 12, 2020

Related U.S. Application Data
(60) Provisional application No. 62/934,141, filed on Nov. 12, 2019.

(51) Int. Cl. G06N 3/08; G16H 20/10; G06K 9/62; G06K 9/66; G06N 5/00; G06N 3/04

(52) U.S. Cl. CPC G06N 3/08; G16H 20/10; G06N 3/04; G06K 9/66; G06N 5/003; G06K 9/6215

(57) ABSTRACT

A system to implement a deep learning network model is disclosed. The system includes machine-readable instructions and data that include a biomedical information library comprising information that includes a plurality of drugs, a plurality of biological targets, a plurality of diseases, and a plurality of adverse effects, a biomedical network system comprising a plurality of networks covering chemical, genomic, phenotypic, and cellular profiles, a score prioritizer to determine new targets for the plurality of drugs based on a concatenation of a low-dimensional vector representation for each drug vertex and each biological target vertex, and a model generator configured to generate a deep learning network model that defines a plurality of relationships between the drugs, the biological targets, and the adverse effects. The plurality of relationships defined by the deep learning network model predict drug target identification and drug repurposing.

### RELATED APPLICATIONS

This application claims priority from U.S. Provisional Application No. 62/934,141, filed 12 Nov. 2019, the subject matter of which is incorporated herein by reference in its entirety.

### STATEMENT REGARDING FEDERALLY SPONSORED RESEARCH

This invention was made with government support under HL138272 and AG066707 awarded by the National Institutes of Health. The government has certain rights in the invention.

### TECHNICAL FIELD

This relates to target identification, or more particularly to a network-based deep learning technology for target identification and drug repurposing.

### BACKGROUND

A biological target is known in the pharmaceutical research industry to characterize the protein or nucleic acid in the body whose activity is changed by a drug resulting in a specific effect. The specified effect may be a desirable therapeutic effect or an unwanted adverse effect. Common drug targets of currently marked drugs include proteins and nucleic acids. Discovery of a medicine involves identifying the biological origin of a disease and the potential targets for intervention. When considering developing a drug to treat a disease, it is important to consider unwanted adverse effects that may arise as a result of administering the drug.

### SUMMARY

In one example, a system to implement a deep learning network model is disclosed. The system includes a memory configured to store machine-readable instructions and data, and a processing unit configured to access the memory and execute the machine-readable instructions. The machine-readable instructions and data include a library comprising information that includes a plurality of drugs, a plurality of biological targets, a plurality of diseases, and a plurality of adverse effects, a biomedical network system comprising a plurality of networks covering chemical, genomic, phenotypic, and cellular profiles, a score prioritizer to determine new targets for the plurality of drugs based on a concatenation of a low-dimensional vector representation for each drug vertex in the biomedical network system, and a low-dimensional vector representation for each biological target vertex in the biomedical network system, and a model generator configured to generate a deep learning network model that defines a plurality of relationships between the drugs, the biological targets, and the adverse effects. The plurality of relationships defined by the deep learning network model offer novel strategies for target identification and drug repurposing.

In another example, a non-transitory computer-readable medium having instructions executable by a processor, the instructions programmed to perform a method to implement a deep learning network model, the A non-transitory computer-readable medium having instructions executable by a processor, the instructions programmed to perform a method to implement a deep learning network model. The method includes retrieving biomedical data from a library, the biomedical data comprising information that includes a plurality of drugs, a plurality of biological targets, a plurality of diseases, and a plurality of adverse effects. The method further includes creating a biomedical network system covering chemical, genomic, phenotypic, and cellular profiles by assembling a plurality of networks. The method further includes generating a low-dimensional vector representation for each drug vertex in the biomedical network system, and generating a low-dimensional vector representation for each biological target vertex in the biomedical network system. The method further includes generating a score prioritizer to determine new targets for the plurality of drugs based on a concatenation of a low-dimensional vector representation for each drug vertex in the biomedical network system, and the low-dimensional vector representation for each biological target vertex in the biomedical network system. The method further includes generating a deep learning network model that defines a plurality of relationships between the drugs, the biological targets, and the adverse effects. The plurality of relationships defined by the deep learning network model predict drug target identification and drug repurposing.

### BRIEF DESCRIPTION OF THE DRAWINGS

FIG. 1 depicts an example network based deep learning system characterized by system of networks.

FIG. 2 depicts example topologies of example drug-target networks.

FIG. 3 illustrates an overview of the process of embedding of target identification networks.

FIG. 4 is a diagram illustrating an example network embedding process.

FIG. 5A illustrates an example bipartite drug-target network.

FIG. 5B is an example known drug-target bipartite network.

FIG. 6 is an example deep learning network model.

FIG. 7A is a graph showing the inhibitory rate of various drugs as predicted by an example deep learning network model.

FIG. 7B is a graph showing the inhibitory rate as a function of topotecan concentration.

FIG. 7C is a graph showing the effect of topotecan on fluorescence intensity.

FIG. 7D is a graph showing the effect of topotecan on cell viability.

FIG. 7E is another graph showing the inhibitory rate of topotecan as a function of concentration

FIG. 7F is a graph illustrating how topotecan alters the variation of circular dichroism (CD) spectrum of RORyt.

FIG. 7G is a graph plotting the circular dichroism (CD) signal for RORγt and RORγt-TPT against wavelength.

FIG. 7H is an illustration showing how topotecan interacts with multiple important residues on human RORyt.

FIG. 8A is a schedule of the application of an experimental autoimmune encephalomyelitis (EAE) model.

FIG. 8B is a graph demonstrating the onset of clinical symptoms after an administration of topotecan.

FIG. 8C is a graph showing weight loss in mice after the application of topotecan during EAE.

FIG. 8D shows a histological analysis of spinal cords after topotecan treatment.

FIG. 8E is an analysis of administration of topotecan reversing fluorescence in EAE mice.

FIG. 8F is an analysis showing a higher accumulation of the fluorescent probe in the brain of vehicle treated mice as compared to the topotecan treatment group.

FIG. 8G is a chart showing that topotecan treatment significantly reduces IL-17 production in brain and spinal cords of EAE mice.

FIG. 9 is an example method to implement a deep learning network model.

### DETAILED DESCRIPTION

Disclosed herein are systems and methods implementing a deep learning network model for identification of drug-target interactions and drug repurposing. The computational systems and methods disclosed herein are used in determining target and potential toxicity of new chemical entities and to identify new chemical starting points for specific disease states. The technology is used to identify candidates, prioritize leads, and predict drug failure.

The system and methods disclosed herein include using different types (e.g., 15) of chemical, genomic, phenotypic, and cellular networks to generate biological and pharmacological relevant features through learning low-dimensional vector representations for both drugs and targets integrating: (1) a deep neural network algorithm for network embedding, which embeds each vertex in a network into a low-dimensional vector space, and (2) a positive-unlabeled-matrix (PU-matrix) completion algorithm. The result is a deep learning network model used to identify target and prioritize leads which curtails the necessity and cost of expensive and time consuming experimental trials. Thus, the systems and methods disclosed herein offer a novel testable hypotheses for systematic, unbiased identification of molecular targets of known drugs.

Pharmaceutical companies spend billions of dollars in the development of a new U.S. Food and Drug Administration (FDA)-approved drug. One of the primary factors for the increased cost is the high failure rate of randomized control trials that are expensive and time-consuming to conduct. The classical hypothesis of ‘one gene, one drug, one disease’ in the conventional drug discovery paradigm most likely contributes to the low success rate in drug development. Without foreknowledge of the complete drug-target network (“polypharmacology”), developing promising strategies for efficacious treatment of multiple complex diseases is challenging, owing to unintended therapeutic effects or multiple drug-target interactions leading to off-target toxicities and suboptimal effectiveness.

Identification of molecular targets for known drugs is essential to improve efficacy while minimizing side effects in clinical trials. However, experimental determination of drug-target interactions is costly and time-consuming. Computational approaches offer novel testable hypotheses for systematic, unbiased identification of molecular targets of known drugs. Recent remarkable advances of omics technologies and systems biology approaches have generated considerable knowledge from chemical, genomic, phenotypic, and cellular networks. A network integrating these makes it possible to infer whether two drugs share a target. For instance, social network-based recommendation algorithms have been adopted for target identification for known drugs, which helps explain side effects and accelerate drug repurposing. However, traditional social network algorithms are based on a single homogeneous drug-target network, and perform poorly on low connectivity (degree) drugs in known drug-target networks. How to efficiently integrate large-scale chemical, genomic, and phenotypic profiles with publicly available systems biology data to accelerate target identification and drug development is an essential task in both academic and industrial communities.

Bioinformatics approaches have offered possibilities for assessment of drug-target interactions and drug-drug relationships. However, several recent studies only focus on using single-dimensional chemoinformatics or bioinformatics data, such as chemical similarities, which limits the accuracy and practical application of the deep learning approaches. The system and method disclosed herein implement deep learning network model for in silico identification of molecular targets for known drugs. The systems and method disclosed herein embeds various types of chemical, genomic, phenotypic, and cellular networks to generate biologically and pharmacologically relevant features through learning low-dimensional but informative vector representations for both drugs and targets. The deep learning network model computationally identifies thousands of novel drug-target interactions with high accuracy, outperforming previously published approaches. The predictions produced by the deep learning network model can be experimentally validated. For example, the experiments demonstrate a potential drug repurposing application in a mouse model of multiple sclerosis based on the deep learning network model prediction. Taken together, if broadly applied, the deep learning network model offers a novel deep learning methodology by exploiting advances in big and diverse biomedical data for accelerating target identification and drug repurposing, minimizing the translational gap in drug development.

FIG. 1 is a block diagram of an example network based deep learning system 100. The system implements a deep learning network model 116. The system 100 includes a memory 102 configured to store machine-readable instructions and data. The system 100 also includes a processing unit configured to access the memory 102 and execute the machine-readable instructions. The machine-readable instructions and data include a biomedical information library 110 having information that includes a plurality of drugs, a plurality of biological targets, a plurality of diseases, and a plurality of adverse effects. In the example shown FIG. 1, the biomedical information library 110 resides in the memory 102 along with the other components of the system. In other examples, the biomedical information library is located separately within another storage medium outside of the memory 102.

The system 100 also includes a biomedical network system 106 comprising a plurality of networks covering chemical, genomic, phenotypic, and cellular profiles. Each of the networks is a graph with a plurality of edges and vertices. The biomedical network system 106 is created using the information from the biomedical information library 110.

The system 100 further includes a new drug-target interactions score prioritizer 118 to identify and rank new drug targets. The new drug-target interactions score prioritizer 118 includes a variety of submodules configured to identify and rank new drug targets. The low-dimensional drug feature matrix learner 120 determines a low dimensional vector representation for each drug vertex of the networks of the biomedical network system 106. The low-dimensional target feature matrix learner determines a low dimensional vector representation for each target vertex of the networks of the biomedical network system 106. The PU matrix completer 124 determines the best projection from the drug space onto the target space such that the projected feature vectors of drugs are geometrically close to the feature vectors of their known interacting targets. The new target identifier 126 infers new targets for a drug ranked by geometric proximity to the projected feature vector of the drug in the projected space.

In all, the score prioritizer 118 determines a prioritized score of new drug-target interactions (DTIs), wherein the prioritized score is determined by the matrix multiplication Sᵢⱼ=XᵢZYⱼ^{T}, wherein X is a matrix representation of drug features, Y is a matrix representation of biological target features, and Z is a positive unlabeled (PU) matrix of a known drug-biological target network that defines known relationships between drugs and biological targets. Detailed aspects of the new drug-target interactions score prioritizer is discussed with reference to FIG. 3.

The system 100 also includes a model generator 112 configured to generate a deep learning network model 116 that defines a plurality of relationships between the drugs, the biological targets, and the adverse effects. The plurality of relationships defined by the deep learning network model predict drug target identification and drug repurposing. The model generator 112 that generates the deep learning network model 116 includes a random surfing model generator 111 configured to capture graph structural information by generating a probabilistic co-occurrence (PCO) matrix for each of the plurality of networks of the biomedical network system. The model generator 112 also includes a pointwise mutual information (PPMI) matrix generator 113 configured to generate a respective PPMI matrix based on each respective PCO matrix. The model generator 112 also includes a stacked denoising autoencoder 115 configured to generate a low-dimensional vector representation of each vertex of a deep learning network model graph. In some examples, the model generator 112 generates a drug vector matrix and a protein vector matrix by network embedding using the t-distributed stochastic neighbor embedding (t-SNE) algorithm.

In some examples, the deep learning network model 116 is a graph comprising a plurality of nodes and a plurality of edges, wherein each node is either a drug, target, or adverse effect, and the edges define interactions between the drugs and targets, and adverse effect reactions to the drugs. In some examples, the interactions between the drugs and targets are novel predicted and validated drug target interactions (DTIs), or predicted and validated DTIs, and wherein the adverse effect reactions to the drugs are either an inferred target-adverse drug reaction (ADR) association, or a clinically reported ADR. Also, in some examples, the deep learning network model 116 is a neural network.

In some examples, the deep learning network model 116 integrates in house biomedical data from companies, such as small molecule-protein networks, drug-disease networks from clinical trials, and omics data (e.g., proteomics and RNA-seq), for target identification or repurposing for clinically failed drugs. The deep learning approach offers powerful tools for target identification and drug repurposing projects for companies by assembling their own in house biomedical data.

In one example, a drug-biological target network can be described as a bipartite graph G (D, T, P), where the drug set denotes as D={d₁, d₂, . . . , dₙ}, target set as T={t₁, t₂, . . . , tₘ}, and interaction set as P={pᵢⱼ: d₁∈D, tⱼ∈T}. An interaction is drawn between dᵢ and tⱼ when drug dᵢ binds with target t₁ with binding affinity (such as IC₅₀, Kᵢ, or K_{d}) less than a given threshold value. Mathematically, a drug-target bipartite network can be presented by an n×m adjacent matrix {pᵢⱼ}, where pᵢⱼ=1 if the binding affinity between dᵢ and tⱼ is less than 10 otherwise pᵢⱼ=0, as described as below.

$${p}_{ij} = \{ \begin{matrix}1 & {IC}_{50} ( {K}_{i} ) \leq 10 μM \\ 0 & {IC}_{50} ( {K}_{i} ) > 10 μM\end{matrix} \qquad Eq . 1$$

Additionally or alternatively, a comprehensive human protein-protein interactome can be assembled using data from bioinformatics and systems biology databases with multiple experimental evidences. Specifically, the interactome is assembled to include high-quality protein-protein interactions (PPIs) with five types of experimental evidences: (i) Binary PPIs tested by high-throughput yeast-two-hybrid (Y2H) systems; (ii) Kinase-substrate interactions by literature-derived low-throughput or high-throughput experiments; (iii) Literature-curated PPIs identified by affinity purification followed by mass spectrometry (AP-MS), Y2H, or by literature-derived low-throughput experiments; (iv) Binary, physical PPIs from protein three-dimensional (3D) structures; and (v) Signaling network by literature-derived low-throughput experiments.

Additionally or alternatively, a drug-drug interaction network can be assembled from data on drug interactions where each drug has the experimentally validated target information. The chemical name, generic name, or commercial name of each drug can be standardized for this purpose can be standardized using available vocabularies. Additionally or alternatively, a drug-disease network can be assembled using known drug indications (i.e., drug-disease associations) from available resources. As with the drug-drug interaction network, the compound name, generic name, or commercial name of each drug and disease can be standardized using available vocabularies. Additionally or alternatively, a drug-adverse network can be assembled from clinically reported drug side effects or adverse drug event (ADE) information by assembling data from available databases. Only ADE data with clinically reported evidence were used, and duplicated drug-ADE associations were excluded.

Additionally or alternatively, a drug-drug similarities network can be assembled from chemical structure information gathered for each drug. In one example, a Tanimoto coefficient can be determined from the molecular access system (MACCS) fragment bit-strings for each drug. The Tanimoto coefficient can range from zero, indicating no bits in common between the two drugs, to one, in which all bits are the same. If two drug molecules have a and b bits set in their MACCS fragment bit-strings, with c of these bits being set in the fingerprints of both drugs, the Tanimoto coefficient (T) of a drug-drug pair is defined as:

$$T = \frac{c}{a + b - c} \qquad Eq . 2$$

Additionally or alternatively, a biological target-biological target network can be assembled from chemical structure information gathered for each drug. Specifically, a protein sequence similarity Sₚ(a, b) of two drug targets a and b can be determined using an alignment algorithm, such as the Smith-Waterman algorithm. The Smith-Waterman algorithm performs local sequence alignment by comparing segments of all possible lengths and optimizing the similarity measure for determining similar regions between two strings of protein canonical sequences of drug targets. The overall sequence similarity, <Sₚ>, of the drug targets binding two drugs A and B is determined by averaging all pairs of proteins a and b with a∈A and b∈B under the condition a≠b. This condition ensures that for drugs with common targets, pairs are not taken into account in which a target would be compared to itself.

$$〈 {S}_{p} 〉 = \frac{1}{{n}_{pairs}} {\sum }_{\{ a , b \}} {S}_{p} ( a , b ) \qquad Eq . 3$$

A gene co-expression network, representing the extent to which drug target-coding genes (a and b) associated with the drug-treated diseases are co-expressed, can be determined by calculating a correlation value, such as Pearson's correlation coefficient (PCC(a, b)), and the corresponding p-value via F-statistics for each pair of drug target-coding genes a and b across various human tissues. In order to reduce the noise of co-expression analysis, PCC(a, b) is mapped into the human protein-protein interactome network to build a co-expressed protein-protein interactome network. The co-expression similarity, <S_{co}>, of the drug target-coding genes associated with two drugs A and B is computed by averaging PCC(a, b) over all pairs of targets a and b with a E A and b E B as below:

$$〈 {S}_{c o} 〉 = \frac{1}{{n}_{pairs}} {\sum }_{\{ a , b \}} \vert PCC ( a , b ) \vert \qquad Eq . 4$$

A gene ontology similarity network, represents a sematic comparison of gene ontology annotations based on biological processes (BP), molecular function (MF), and cellular component (CC), excluding annotations inferred computationally. This provides quantitative ways to compute similarities between genes and gene products. Gene ontology similarity, S_{GO}(a, b), is calculated for each pair of drug target-coding genes a and b using a graph-based semantic similarity measure algorithm. The overall GO similarity, <S_{GO} (a, b)> of the drug target-coding genes binding to two drugs A and B is determined by Eq. 5, which averages all pairs of drug target-coding genes a and b with a∈A and b∈B.

$$〈 {S}_{GO} 〉 = \frac{1}{{n}_{pairs}} {\sum }_{\{ a , b \}} {S}_{GO} ( a , b ) \qquad Eq . 5$$

A drug-drug clinical similarity network can be derived from the drug Anatomical Therapeutic Chemical (ATC) classification systems codes. The kth level drug clinical similarity (Sₖ) of drugs A and B is defined via the ATC codes as below.

$${S}_{k} ( A , B ) = \frac{A T {C}_{k} ( A ) ⋂ {ATC}_{k} ( B )}{A T {C}_{k} ( A ) ⋃ {ATC}_{k} ( B )} \qquad Eq . 6$$

where ATCₖ represents all ATC codes at the kth level. A score S_{atc}(A, B) is used to define the clinical similarity between drugs A and B:

$${S}_{atc} ( A , B ) = \frac{{\sum }_{k = 1}^{n} {S}_{k} ( A , B )}{n} \qquad Eq . 7$$

where n represents the five levels of ATC codes (ranging from 1 to 5). Note that drugs can have multiple ATC codes. For example, nicotine (a potent parasympathomimetic stimulant) has four different ATC codes: N07BA01, A11HA01, C04AC01, C10AD02. For a drug with multiple ATC codes, the clinical similarity was computed for each ATC code, and then, the average clinical similarity was used.

A disease-gene network can utilize disease-gene annotation data from various bioinformatics data sources to annotate all protein-coding genes using gene Entrez ID, chromosomal location, and the official gene symbols from the National Center for Biotechnology Information (NCBI) database.

A network embedding algorithm is used to extract low dimensional representations of vertexes in networks, aiming to capture and preserve the network structure. The low-dimensional vectors obtained from this process encode the relevant biological properties, association information, and topological context of each drug (or target) node in the heterogeneous drug-target-disease network. In one example, network embedding is performed via a Deep Neural Networks for Graph Representation (DNRG) embedding model. The DNRG model utilizes a random surfing model to capture network information and generates a probabilistic co-occurrence matrix and calculates a positive pointwise mutual information (PPMI) matrix based on the probabilistic co-occurrence matrix by following. After that, a stacked denoising autoencoder is used to learn low-dimensional vertex representations and model non-linearities.

During random surfing, the vertices of a network are first ordered randomly. Assuming a current vertex is the i-th vertex, a transition matrix A captures the transition probabilities between different vertices. In this example, a random surfing model with restart is used, which introduces a pre-defined restart probability at the initial node for every iteration. It takes both local and global topological connectivity patterns within the network into consideration to fully exploit the underlying direct or indirect relations between nodes. Thus, at each time, there is a probability a that the random surfing procedure will continue, and a probability 1−α that it will return to the original vertex and restart the procedure, which can be diagonalized as follow:

pₖ=α·pₖ₋₁A+(1−α)p₀ Eq. 8

where pₖ is a row vector, whose j-th entry indicates the probability of reaching the j-th vertex after k steps of transitions, and p₀ is the initial 1-hot vector with the value of the i-th entry being 1 and all other entries being 0. The random surfing step yields a probabilistic co-occurrence matrix.

After yielding the probabilistic co-occurrence matrix, the PPMI matrix is calculated. The PPMI matrix can be viewed as a matrix factorization method which factorizes a co-occurrence matrix to yield network representations. The PPMI matrix can be constructed as follows:

$$PPMI = max ( log \frac{M ( i , j ) * {\sum }_{i}^{{N}_{d}} {{\sum }_{j}}^{{N}_{t}} M ( i , j )}{{\sum }_{i}^{{N}_{d}} M ( i , j ) * {{\sum }_{j}}^{{N}_{t}} M ( i , j )} , 0 ) \qquad Eq . 9$$

where M is the original co-occurrence matrix, N_{d} is the drug number, Nₜ is the target number. Each negative value is assigned to 0.

Finally, a stacked denoising autoencoder (SDAE) is used to generate compressed, low-dimensional vectors from the original high-dimensional vertex vectors. This process essentially performs dimension reduction that maps data from a high dimensional space into a lower dimensional space. Denoising autoencoders partially corrupt the input data before taking the training step, adding noise helps a SDAE to learn features that are robust to partial corruption of input data. Specifically, each input sample x (a vector) is corrupted randomly by assigning the entries in the vector to 0 with a certain probability. This idea is analogous to that of modeling missing entries in matrix completion tasks, where the goal is to exploit regularities in the data matrix to effectively recover the complete matrix under certain assumptions. A SDAE model minimizes the regularized problem and tackles reconstruction error, defined as follows:

$${min}_{\{ {w}_{l} \} , \{ {b}_{l} \}} {‖ x - \hat{x} ‖}_{F}^{2} + λ {\sum }_{l} {‖ {W}_{l} ‖}_{F}^{2} \qquad Eq . 10$$

where L is the number of layers, Wₗ is weight matrix and bₗ is bias vector of layer l∈{1, . . . , L} which can be learned by back-propagation algorithm. λ is a regularization parameter and ∥⋅∥_{F} denotes the Frobenius norm. The first L/2 layers of the model act as an encoder, and the last L/2 layers act as a decoder. The middle layer is the key that enables SDAE to reduce dimensionality and extract effective representations of side information.

In practice, only positive associations between drugs and targets are observed, which means no “negative” entries are sampled. Consequently, a positive-unlabeled (PU) learning framework is utilized, where observed and unobserved entries are penalized differently in the objective. Assume the drug-target associations matrix is given as P∈ [symbol] ^{N}^{_{d}}^{×N}^{ₜ} where N_{d} is the number of drugs and Nₜ is the number of targets. When Pᵢⱼ=1, infers drug i is linked to target j while zero indicates the relationship is unobserved. After the feature extraction process, a decomposing function is constructed to recover a low-rank matrix Z∈ [symbol] ^{f}^{_{d}}^{×f}^{ₜ} from the known associations matrix P with the form of Z=WH^{T}, where W∈ [symbol] ^{f}^{_{d}}^{×k} and H∈ [symbol] ^{f}^{ₜ}^{×k}, k<<N_{d}, Nₜ. The optimization problem of our model is parameterized as:

$${min}_{W , H} {\sum }_{( i , j ) ∈ {Ω}^{+}} {( {P}_{i j} - {x}_{i} W {H}^{T} {y}_{j}^{T} )}^{2} + α {\sum }_{( i , j ) ∈ {Ω}^{-}} {( {P}_{ij} - {x}_{i} W {H}^{T} {y}_{j}^{T} )}^{2} + λ ( {‖ W ‖}_{F}^{2} + {‖ H ‖}_{F}^{2} ) \qquad Eq . 11$$

where the set Ω includes both positive and negative entries, such that Ω=Ω⁺∪Ω⁻, let Ω⁺ denotes the observed samples and Ω⁻ denotes the missing entries chosen as negatives.

For biased inductive matrix completion, the value α is the key parameter, which determines the penalty of the unobserved entries toward zero. α is set to a value less than one, because the penalty weights for observed entries should be greater than the missing ones. The biased value α and regulation parameter λ are selected over the grid search. Then, the likelihood of the pairwise interaction score between drug i and target j is approximated as:

score(i,j)=xᵢWH^{T}yⱼ^{T} Eq. 12

where the higher score means a higher possibility that drug i is correlated with target j.

For the homogeneous interaction networks (e.g., drug-drug interaction network) and similarity networks (e.g., drug chemical similarity network), the feature representation of each drug or target is generated by directly running the DNGR model on each of these networks. For the association networks, e.g., drug-disease, drug-side-effect, and protein-disease networks, the corresponding similarity networks are constructed based on the Jaccard similarity coefficient, and then run the DNGR model on these similarity networks. Taking the drug-disease association network as an example, we use the following formula to measure the similarity between drug i and drug j:

$$S i m ( i , j ) = \frac{\vert {Disease}_{i} ⋂ {Disease}_{j} \vert}{\vert {Disease}_{i} ⋃ {Disease}_{j} \vert} \qquad Eq . 13$$

Where Diseaseᵢ denotes the set of diseases of drug i. Then we run the DNGR model on this similarity network to obtain the feature representation of drugs. In the same manner, the similarity networks for proteins can be constructed.

As shown in FIG. 1, the system 100 also includes a command interface 104 to issue commands to build the biomedical network system, and actuate the model generator. The command interface can be a text editor enabling a user to build the biomedical network system 106 using a programming language.

FIG. 2 depicts example topologies of example drug-target networks, such as the networks 107 that make up the network system 106 of FIG. 1. The networks shown in FIG. 2 implement a network model for the identification of drug-target interactions and drug repurposing. In the example shown in FIG. 2, there are two classifications of networks, namely chemical and phenotypic networks 202, and genomic and cellular networks 212. Within the chemical and phenotypic networks 202 are one or more drug-drug networks 204, one or more drug-disease networks 206, one or more drug-side-effect networks 208, and one or more drug similarities networks 210. The drug-similarities networks 210 model various similarities, including chemical similarity, therapeutic similarity, protein sequence similarity, biological process similarity, cellular component similarity, and molecular function similarity. Within the genomic and cellular networks 212 are one or more protein-protein networks 214, one or more protein-disease networks 216, and one or more target (protein)-similarities networks 218. The target (protein)-similarities networks 218 model various similarities, including protein sequence similarity, biological process similarity, cellular component similarity, and molecular function similarity. Also included among the networks are one or more drug-protein 220 networks. As used herein, “target” and “protein” are used interchangeably, though other types of target network modeling is possible, for example including when the targets are nucleic acids.

The biomedical networks shown in FIG. 2 are first created. After one or more networks are created, the networks are embedded into the deep neural network model to learn a low-dimensional but informative vector representation for both drugs and targets. In one example, a low-dimensional but informative vector representation for both drugs and targets is learned using a deep neural network for graph representations algorithm. In one example, the system network covers four classical druggable target families, namely G-protein-coupled receptors (GPCRs), kinases, nuclear receptors and ion channels. In one example, included in the network are 4.978 experimentally validated drug-target interactions connected 732 approved drugs and 1,815 human targets, by assembling binding affinity from different data resources (e.g., six data resources). Using t-SNE (t-distributed stochastic neighbor embedding), a nonlinear dimensionality reduction method, the projected drugs grouped by the first-level of the Anatomical Therapeutic Chemical classification system code in 2D space. In one example, known drugs fell into the same 2D space when plotted against either therapeutic indication or target family. After learning the low-dimensional feature vectors, the optimization is modified compared to low-rank matrix completion. For any given drug-target pair, it is difficult to verify unobserved evidence that such a connection is indeed nonexistent, or hidden, owing to lack of reported negative samples from publicly available literatures. Thus, a PU learning formulation to low-rank matrix completion is employed, which is able to infer whether two drugs share a target.

FIG. 3 illustrates an overview of the process of embedding of target identification networks. The deep learning network system (such as the deep learning network system 100 of FIG. 1) embeds the one or more different types of chemical and phenotypic networks 202 and the genomic and cellular networks 212. During the embedding process 300, the deep learning network system applies an algorithm (e.g., a neural network algorithm) to learn a low-dimensional vector representation of the features for each node. For example, a feature matrix is learned for drugs (e.g., X), and a feature matrix is learned for targets (e.g., Y^{T}). FIG. 3 shows an example drug feature matrix 302 (X), and an example target (protein) feature matrix 304 (Y^{T}). Each row Xᵢ in the drug feature matrix 302 represents the feature vector of a drug, and each row Yⱼ in the target feature matrix 304 (or column in the transposed target feature matrix Y^{T}) represents the feature vector of a target. In FIG. 3, there are N_{d} rows in the drug feature matrix 302, each row Xᵢ corresponding to a drug (e.g., there are N_{d} drugs), and there are N_{g} columns in the transpose of the target feature matrix 304 (Y^{T}), each column corresponding to a protein (e.g., there are N_{g} proteins). Each row Xᵢ within the drug feature matrix 302 X has f_{d} features (e.g., each drug has f_{d} features), and each column in the transposed target feature matrix 304 Y^{T} has f_{g} features (e.g., each protein has f_{g} features). The deep learning network system applies PU-matrix completion 306 to find the best projection from the drug space onto the target (protein) space, such that the projected features vectors of drugs are geometrically close to the feature vectors of their known interacting targets. The PU-matrix completion 306 is performed by the matrix multiplication Z=WH^{T}, where W has dimensions f_{d} by k, and H^{T} has dimensions k by f_{g}. The deep learning network system then infers new targets for a drug ranked by geometric proximity to the projected feature vector of the drug in the projected space. The new target inference is represented by the matrix multiplication Sᵢⱼ=XᵢZYⱼ^{T}, where Sᵢⱼ is a score prioritizer of new drug-target interactions.

FIG. 4 is a diagram illustrating an example network embedding process 300. The deep learning network for graph representations model (such as the deep learning network for graph representations model 108 of FIG. 1) is constructed from three major network embedding steps: 1) creating a random surfing mode to capture the graph structural information and generate a probabilistic co-occurrence (PCO) matrix, 2) calculation of a shifted positive pointwise mutual information (PPMI) matrix based on the PCO matrix, and 3) creating a stacked denoising autoencoder to generate compressed, low-dimensional vectors from the original high dimensional vertex vectors. The learned low-dimensional feature vectors encode the relational properties, association information and topological context of each node in the heterogeneous drug-gene-disease network.

Thus, FIG. 4 shows networks 407, where there are k networks (k being a positive integer). Among the networks 407 shown in FIG. 4 is a drug-drug network 407a, a protein-disease network 407b, and a protein-protein network 407k. A PCO matrix is created for each of the networks 407, namely PCO matrix 409a, PCO matrix 409b, and PCO matrix 409k. A PPMI matrix is calculated based on each of the PCO matrices. Thus the PPMI matrix 411a is created based on the PCO matrix 409a, the PPMI matrix 411b is created based on the PCO matrix 409b, and the PPMI matrix 411k is created based on the PCO matrix 409k. Stacked denoising autoencoders 413 are created from the PPMI matrices 411. Thus the stacked denoising autoencoder 413a is created from the PPMI matrix 411a, the stacked denoising autoencoder 413b is created from the PPMI matrix 413b, and the stacked denoising autoencoder 413k is created from the PPMI matrix 413k. The stacked denoising autoencoders 413 are used to generate compressed, low-dimensional vectors 415 from the original high dimensional vertex vectors. The learned low-dimensional feature vectors (415a, 415b, and 415k) encode the relational properties, association information, and topological context of each node in the heterogeneous drug-gene-disease network 408. The heterogeneous drug-gene-disease network 408 is the resulting deep learning network for graph representations model (such as deep learning network for graph representations model 108 of FIG. 1).

FIG. 5A illustrates an example computationally predicted bipartite drug-target network 500 covering novel predicted drug-target interactions across four target families 522. FIG. 5B is an example known drug-target bipartite network 550 covering four types of druggable targets 524. To uncover new targets for known drugs, the top novel drug-target interactions predicted by the deep learning network for graph representations model are prioritized. In FIG. 5A, the four target families are GPCRs, kinases, NRs, and ICs. Two-thousand two hundred and fourteen (2,214) interactions are computationally identified, the 2,214 interactions connecting 79 GPCRs and 732 known drugs based on the top five predicted candidates by the deep learning network for graph representations model. Compared to the known drug-target bipartite network 550, the computationally predicted drug-target interactions shown by the bipartite drug-target network 500 show strong polypharmacology for FDA-approved drugs.

FIG. 6 is an example deep learning network model 600 showing the relationships between drugs, targets, and adverse events. The deep learning network model is constructed as a graph, where each node is either a drug, target or adverse effect. The edges of the deep learning network model graph represent either a novel predicted and validated drug target interaction (DTI), a predicted and validated DTI, an inferred biological target-adverse drug reaction (ADR) association, or a clinically reported ADR. The model 600 explains the mechanism-of-action of known drugs for characterizing their adverse or therapeutic effects. Dobutamine, shown as a drug node in the model 600, is an approved sympathomimetic drug used in the treatment of heart failure and cardiogenic shock by targeting beta1-adrenergic receptors. Studies have shown, and the model 600 also shows, that dobutamine leads to several types of cardiovascular complications, such as palpitations, bradycardia, and hypertension. Via the deep learning network for graph representations model 600, it is determined that dobutamine has interactions with several additional GPCRs, including DRD1, DRD2, DRD3, ADRA2A, and ADRA2B. Some of these predictions (e.g., DRD1, DRD2, DRD3, ADRA2A, and ADRA2B) are validated by published experimental data, including two novel predictions: ADRA2A (IC50=10.83 μM) and DRD2 (IC50=8.22 μM). Genetic studies showed that ADRA2A played a critical role in the regulation of systemic sympathetic activity and cardiovascular responses such as heart rate and blood pressure. Thus, the identified off-targets, such as ADRA2A and ADRA2B, may help explain the cardiovascular complications associated with dobutamine treatment. Alosetron (a selective serotonin type-3 receptor antagonist) and tegaserod (a 5-hydroxytryptamine receptor-4 agonist) have been approved for the management of severe diarrhea-predominant irritable bowel syndrome in women. Subsequently, both drugs were withdrawn from the market due to a potential risk of ischemic colitis and several adverse cardiovascular effects, such as angina pectoris. Multiple polymorphisms on HTR2A, HTR1A, HTR2B, and HTR3C were detected in patients with high blood pressure, metabolic syndrome, and obstructive sleep apnea syndrome. The deep learning network for graph representations model 600 computationally identifies several validated off-targets for alosetron and tegaserod, which may help understand the molecular mechanisms of several adverse effects, such as sleep disorder and angina pectoris. Collectively, the molecular targets identified by deep learning network for graph representations model 600 offer new mechanism-of-action for characterizing adverse effects of known drugs. The identified novel molecular targets for known drugs by deep learning network for graph representations model 600 also offer new possibilities for treating new human diseases (e.g., drug repurposing). Testing the top novel candidates prioritized by the deep learning network model shows that the identified drugs are promising. In an example, the deep learning network model is used to screen a library of repurposed drugs for activity against targets linked to MS.

FIG. 7A is a graph showing the inhibitory rate of various drugs as predicted by an example deep learning network system. The deep learning network model predicted 18 drugs (shown in FIG. 7A) would have activity and when experimentally screened against RORγt, 6 had inhibitory activity >30%, with topotecan being the most active. In total, 18 purchasable drugs for RORγt were tested using a cell-based luciferase reporter assay. In this assay, GAL4-RORγt, with fused human RORγt LBD and GAL4 DNA binding domain are co-transfected into HEK293T cells with a luciferase reporter gene harboring the GAL4 response element33. As shown in FIG. 7A, among 18 deepDTnet-predicted drugs, six drugs, including tazarotene, norethindrone, rosiglitazone, bezafibrate, topotecan and spironolactone, have an inhibitory rate higher than 30% on human RORγt at a concentration of 10 μM. Topotecan is shown as the most potent inhibitor of RORγt with an inhibitory rate of 71.0% at 10 μM.

FIG. 7B is a graph showing the inhibitory rate as a function of topotecan concentration. As shown in FIG. 7B, topotecan exhibits a dose-dependent antagonistic activity with an IC₅₀ value of 0.43±0.02 μM in GAL4-RORγt expressing HEK293T cells. FIG. 7C is a graph showing the effect of topotecan on fluorescence intensity. FIG. 7C shows that no suppression is observed in the control firefly luciferase activity experiments, indicating that topotecan has no promiscuous effects on luciferase. FIG. 7D is a graph showing the effect of topotecan on cell viability. FIG. 7D shows that topotecan has a minor effect on HEK293T cell viability at the same concentration range in the reporter assay, revealing tolerable toxic profiles in normal human cells. As topotecan is the most potent compound in the luciferase reporter assay, we selected it for further experimental validation.

Nuclear receptors execute their versatile transcriptional functions by recruiting positive and negative regulatory proteins, known as coactivators or corepressors, respectively. Agonists promote interactions between nuclear receptors and coactivators, while antagonists either inhibit coactivator binding or facilitate corepressor recruitment. The functional change of the binding of topotecan on RORγt is investigated by utilizing a HTRF assay to evaluate ligand-induced coactivator recruitment to RORγt.

FIG. 7E is another graph showing the inhibitory rate of topotecan as a function of concentration as predicted by an example deep learning network system. As shown in FIG. 7E, topotecan disrupts the interaction of RORγt-LBD with steroid receptor coactivator-1 (SRC-1) cofactor peptide in a dose-dependent manner with an IC₅₀ value of 6.65±0.02 μM. The HTRF-based coactivator recruitment results indicate that topotecan directly bind to RORγt and regulate the interaction between RORγt and SRC-1 peptide by inducing a conformational change of RORγt.

FIG. 7F is a graph illustrating how topotecan alters the variation of circular dichroism (CD) spectrum of RORγt. CD is a powerful method for probing protein and ligand interactions in solution. As shown in FIG. 7F, topotecan alters the variation of CD spectrum of RORγt, confirming the direct binding of topotecan on RORγt-LBD. FIG. 7G is a graph plotting CD Signal as a function of wavelength. As shown in FIG. 7G, high-performance liquid chromatography (HPLC) further indicates that topotecan directly interacts with RORγt-LBD, but not with retinoid X receptor α LBD (RXRα-LBD). FIG. 7H is an illustration showing how topotecan interacts with multiple important residues on human RORγt. The binding mode of topotecan to human RORγt is examined using molecular docking. FIG. 7H shows that topotecan interacts with multiple important residues on human RORγt, such as Arg364, Met365, Gln286, and Glu379. Specifically, topotecan shows a direct hydrogen-bonding interaction with Gln286, which is consistent with previously experimental studies. Put together, by combining information gleaned from the deep learning network model and experimental assays, topotecan is identified as a novel, direct inhibitor of human RORγt.

Continuing with the analysis of topotecan as predicted to be effective by the deep learning network model, it is shown that topotecan reverses multiple sclerosis in vivo. Multiple sclerosis is an inflammatory-mediated demyelinating disease of the central nervous system (CNS) and the major cause of non-traumatic neurological disability in young adults with soaring costs. Three principles are adhered to when analyzing the effectiveness of topotecan on multiple sclerosis: (1) topotecan directly inhibits human RORγt, as identified by the deep learning network model and multiple complementary assays (FIGS. 7A-7F); (2) RORγt emerged as a key target for treatment of multiple sclerosis; and (3) topotecan has been shown to have ideal pharmacokinetics in the context of the neurological diseases (i.e., blood-brain barrier) and can be investigated for treatment of Angelman syndrome based on a preclinical model.

Experimental data has shown that topotecan has very low toxicity on normal cell lines or other mice organs even though topotecan is a chemotherapeutic agent. In addition, as shown in Tables 1 and 2 below, topotecan has exhibited ideal brain penetration and other pharmacokinetic properties in mouse models as well. Accordingly, as predicted by the systems and methods presented herein, topotecan is a good drug candidate in possible treatment of MS.

TABLE 1
Pharmacokinetic parameters of topotecan
after intraperitoneal injection in mice

| | Tₘₐₓ | Cₘₐₓ | AUC₀₋ₜ | AUC_{0-inf} | t_{1/2} |
|---|---|---|---|---|---|
| | (h) | (ng/mL) | (ng*h/mL) | (ng*h/mL) | (h) |
| Brain | 0.5 | 121.29 | 169.53 | 172.74 | — |
| Plasma | 0.5 | 3254.57 | 3276.16 | 3309.18 | 4.81 |

Where Cₘₐₓ is the peak concentration, Tmax is the time between administration and reaching Cₘₐₓ, and t_{1/2} is the elimination half-life.

TABLE 2
Concentration of T0901317 in mice brain samples

| Con (ng/g) | Vehicle | TPT | P-value |
|---|---|---|---|
| 1 | 4214.45 | 2788.48 | 0.0029 |
| 2 | 5433.60 | 4396.96 | |
| 3 | 3964.91 | 3365.97 | |
| 4 | 4506.24 | 3736.16 | |
| 5 | 4013.55 | 3212.96 | |
| Mean | 4426.55 | 3500.11 | |
| SD | 601.91 | 605.58 | |
| RSD | 0.14 | 0.17 | |

In the data presented in Table 2, T0901317, an orthosteric ligand of RORγt, was used as the tracer for assessing target occupancy of TPT. A Student's t-test was performed to obtain the p-value, and sterile water was used as vehicle.

FIG. 8A is a schedule of the application of an experimental autoimmune encephalomyelitis (EAE) model. EAE is a most frequently used experimental animal model for human multiple sclerosis. To investigate the therapeutic potential of topotecan in multiple sclerosis, EAE was induced in C57BL/6 mice by active immunization with MOG33-55 in complete Freund's adjuvant (CFA) followed by pertussis toxin administration, according to the application schedule shown in FIG. 8A. Topotecan (10 mg/kg) or vehicle (sterile water, control) was administered intraperitoneally every four days during the course of EAE. Disease severity was assessed and graded using a five-point scoring system for 15 days.

FIG. 8B is a graph demonstrating the onset of clinical symptoms after an administration of topotecan. FIG. 8B shows that the administration of topotecan leads to a significantly delay in the onset of clinical symptoms and an observable reduction of clinical score of the EAE mice. During the course of EAE, changes in body weight also reflect disease severity. FIG. 8C is a graph showing weight loss in miss after the application of topotecan during EAE. FIG. 8C shows that mice treated with topotecan are more tolerable to EAE induced body weight loss than vehicle-treated mice. FIG. 8D shows a histological analysis of spinal cords after topotecan treatment. FIG. 8D shows a histological analysis of spinal cords of the EAE mice conducted on day 20 after immunization. Hematoxylin and eosin (H&E) staining shows significant infiltration of leukocytes in the spinal cord tissues from vehicle-treated mice, whereas infiltration is largely reduced following topotecan treatment. Luxol fast blue (LFB) staining shows severe demyelination in white matter of EAE mice, whereas demyelination is significantly reversed in topotecan treated mice.

Multiple sclerosis is a chronic demyelinating disease accompanied by blood-brain barrier disruption. Near-infrared in vivo imaging is further utilized to evaluate the demyelination and blood-brain barrier leakage in EAE mice. A near-infrared fluorescent dye, 3, 3′-diethylthiatricarbocyanine iodide (DBT), easily enter into the brain and selectively binds to myelin fibers45. FIG. 8E demonstrates that administration of topotecan reverses fluorescence in EAE mice. Cy5.5-BSA, a fluorescent BSA conjugate with bright near infrared fluorescence, penetrates the brain when the blood-brain barrier is disrupted. FIG. 8F demonstrates a higher accumulation of the fluorescent probe in the brain of vehicle treated mice as compared to the topotecan treatment group. FIG. 8F shows a higher accumulation of the fluorescent probe in the brain of vehicle treated mice as compared to the topotecan treatment group.

T helper 17 (Th17) cells are a highly pro-inflammatory lineage of T helper cells defined by their production of interleukin 17 (IL-17)46. RORγt is necessary and sufficient for cytokine IL-17 expression in mouse and human Th17 cells46. Given the inhibitory effects of topotecan against RORγt, it was investigated whether topotecan affects IL-17 expression in EAE mice. Accordingly, FIG. 8G is a chart showing that topotecan treatment significantly reduces IL-17 production in brain and spinal cords of EAE mice. Of note, ELISA experiments revealed that topotecan treatment significantly reduces IL-17 production in brain and spinal cords of EAE mice (as shown in FIG. 8G). In summary, the results demonstrate that topotecan alleviates the clinical symptoms of the EAE model. Although potential off-target effects and clinical trials would remain areas of further investigation, the findings of FIG. 8A-8G show that topotecan identified by the deep learning network model offers a potential therapeutic strategy for multiple sclerosis by targeting RORγt.

FIG. 9 is an example method 900 to implement a deep learning network model. At 902, biomedical data is retrieved from a library. The biomedical data includes information that includes a plurality of drugs, a plurality of biological targets, a plurality of diseases, and a plurality of adverse effects.

At 904, a biomedical network system is created by assembling a plurality of networks, the biomedical network system covering chemical, genomic, phenotypic, and cellular profiles. Creating the biomedical network system includes assembling a plurality of graph networks, the graph networks including at least one drug-drug network that defines relationships between the drugs, at least one drug-disease network that defines relationships between the drugs and the diseases, at least one drug-adverse effect network that defines relationships between the drugs and the adverse effects, at least one drug similarities network that defines drug similarities between the drugs, at least one biological target-biological target network that defines relationships between the biological targets, at least one biological target-disease network that defines the relationships between the biological targets and the diseases, at least one biological target similarities network that defines biological target similarities between the biological targets, and at least one drug-biological target network that defines relationships between the drugs and biological targets. Each of the plurality of graph networks comprises at least one vertex, and at least one edge connects two vertices. The drug similarities include chemical similarities, therapeutic similarities, protein sequence similarities, biological process similarities, cellular component similarities, and molecular function similarities, and the biological target similarities include protein sequence similarities, biological process similarities, cellular component similarities, and molecular function similarities.

At 906, a low-dimensional vector representation for each drug vertex in the biomedical network system is generated. At 908, a low-dimensional vector representation for each biological target vertex in the biomedical network system is generated. At 910, a score prioritizer is generated, wherein the score prioritizer determines new targets for the plurality of drugs based on a concatenation of the a low-dimensional vector representation for each drug vertex in the biomedical network system, and the low-dimensional vector representation for each biological target vertex in the biomedical network system. The score prioritizer determines a score of new drug-target interactions (DTIs), wherein the prioritized score is determined by the matrix multiplication Sᵢⱼ=XᵢZYⱼ^{T}, wherein X is a matrix representation of drug features, Y is a matrix representation of biological target features, and Z is a positive unlabeled (PU) matrix of a known drug-biological target network that defines known relationships between drugs and biological targets.

At 912, a deep learning network model that defines a plurality of relationships between the drugs, the biological targets, and the adverse effects is generated. The plurality of relationships defined by the deep learning network model predict drug target identification and drug repurposing. In some examples, the deep learning network model is a graph comprising a plurality of nodes and a plurality of edges, wherein each node is either a drug, target, disease, or adverse effect, and the edges define interactions between the drugs and targets, and adverse effect reactions to the drugs. Furthermore, the interactions between the drugs and targets are novel predicted and validated drug target interactions (DTIs), or predicted and validated DTIs, and wherein the adverse effect reactions to the drugs are either an inferred target-adverse drug reaction (ADR) association, or a clinically reported ADR.

Generating the deep learning network model includes generating a random surfing model configured to capture graph structural information by generating a probabilistic co-occurrence (PCO) matrix for each of the plurality of networks of the biomedical network system, generating a respective PPMI matrix based on each respective PCO matrix, and creating a stacked denoising autoencoder to generate a low-dimensional vector representation of each vertex of a deep learning network model graph. In some examples, generating the deep learning network model includes generating a drug vector matrix and a protein vector matrix by network embedding using the t-distributed stochastic neighbor embedding (t-SNE) algorithm. In some examples, the deep learning network model is a neural network. In some examples, the method 900 further includes issuing commands to actuate the model generator using a command interface.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

What is claimed is:

1. A system to implement a deep learning network model, comprising:
a memory configured to store machine-readable instructions and data;
a processing unit configured to access the memory and execute the machine-readable instructions, the machine-readable instructions and data comprising:
a biomedical information library comprising information that includes a plurality of drugs, a plurality of biological targets, a plurality of diseases, and a plurality of adverse effects;
a biomedical network system comprising a plurality of networks covering at least one of chemical, genomic, phenotypic, and cellular profiles;
a score prioritizer to determine new targets for the plurality of drugs based on a concatenation of a low-dimensional vector representation for each drug vertex in the biomedical network system, and a low-dimensional vector representation for each biological target vertex in the biomedical network system; and
a model generator configured to generate a deep learning network model that defines a plurality of relationships between the drugs, the biological targets, and the adverse effects from the plurality of networks;
wherein the plurality of relationships defined by the deep learning network model predict drug target identification and drug repurposing.

2. The system of claim 1, wherein the plurality of networks of the biomedical network system comprise a plurality of graph networks, the plurality of graph networks including:
at least one drug-drug network that defines relationships between the drugs;
at least one biological target-biological target network that defines relationships between the biological targets; and
at least one drug-biological target network that defines relationships between the drugs and biological targets;
wherein each of the plurality of graph networks comprises at least one vertex, and at least one edge connects two vertices.

3. The system of claim 2, wherein the plurality of graph networks further include:
at least one drug-disease network that defines relationships between the drugs and the diseases;
at least one drug-adverse effect network that defines relationships between the drugs and the adverse effects;
at least one drug similarities network that defines drug similarities between the drugs;
at least one biological target-disease network that defines the relationships between the biological targets and the diseases; and
at least one biological target similarities network that defines biological target similarities between the biological targets.

4. The system of claim 1, wherein the deep learning network model is a graph comprising a plurality of nodes and a plurality of edges, wherein each node is either a drug, target, or adverse effect, and the edges define interactions between the drugs and targets, and adverse effect reactions to the drugs.

5. The system of claim 4, wherein the interactions between the drugs and targets are novel predicted and validated drug target interactions (DTIs), or predicted and validated DTIs, and wherein the adverse effect reactions to the drugs are either an inferred target-adverse drug reaction (ADR) association, or a clinically reported ADR.

6. The system of claim 1, wherein the model generator generates a drug vector matrix and a protein vector matrix by network embedding using the t-distributed stochastic neighbor embedding (t-SNE) algorithm.

7. The system of claim 1, wherein the model generator that generates the deep learning network model comprises:
a random surfing model generator configured to capture graph structural information by generating a probabilistic co-occurrence (PCO) matrix for each of the plurality of networks of the biomedical network system;
a pointwise mutual information (PPMI) matrix generator configured to generate a respective PPMI matrix based on each respective PCO matrix; and
a stacked denoising autoencoder configured to generate a low-dimensional vector representation of each vertex of a deep learning network model graph.

8. The system of claim 1, wherein the score prioritizer determines a prioritized score of new drug-target interactions (DTIs), wherein the prioritized score is determined by the matrix multiplication Sᵢⱼ=XᵢZYⱼ^{T}, wherein X is a matrix representation of drug features, Y is a matrix representation of biological target features, and Z is a positive unlabeled (PU) matrix of a known drug-biological target network that defines known relationships between drugs and biological targets.

9. The system of claim 1, wherein the deep learning network model is a neural network.

10. The system of claim 1, further comprising a command interface configured to issue commands to build the biomedical network system, and actuate the model generator.

11. A non-transitory computer-readable medium having instructions executable by a processor, the instructions programmed to perform a method to implement a deep learning network model, the method comprising:
retrieving biomedical data from a library, the biomedical data comprising information that includes a plurality of drugs, a plurality of biological targets, a plurality of diseases, and a plurality of adverse effects;
creating a biomedical network system covering at least one of chemical, genomic, phenotypic, and cellular profiles by assembling a plurality of networks;
generating a low-dimensional vector representation for each drug vertex in the biomedical network system;
generating a low-dimensional vector representation for each biological target vertex in the biomedical network system;
generating a score prioritizer to determine new targets for the plurality of drugs based on a concatenation of the a low-dimensional vector representation for each drug vertex in the biomedical network system, and the low-dimensional vector representation for each biological target vertex in the biomedical network system; and
generating a deep learning network model that defines a plurality of relationships between the drugs, the biological targets, and the adverse effects;
wherein the plurality of relationships defined by the deep learning network model predict drug target identification and drug repurposing.

12. The non-transitory computer readable medium of claim 11, wherein creating the biomedical network system comprises assembling a plurality of graph networks, the plurality of graph networks including:
at least one drug-drug network that defines relationships between the drugs;
at least one drug-disease network that defines relationships between the drugs and the diseases;
at least one drug-adverse effect network that defines relationships between the drugs and the adverse effects; and
at least one drug similarities network that defines drug similarities between the drugs;
wherein each of the plurality of graph networks comprises at least one vertex, and at least one edge connects two vertices.

13. The non-transitory computer readable medium of claim 12, wherein the plurality of graph networks further include:
at least one biological target-biological target network that defines relationships between the biological targets;
at least one biological target-disease network that defines the relationships between the biological targets and the diseases;
at least one biological target similarities network that defines biological target similarities between the biological targets; and
at least one drug-biological target network that defines relationships between the drugs and biological targets.

14. The non-transitory computer readable medium of claim 11, wherein the deep learning network model is a graph comprising a plurality of nodes and a plurality of edges, wherein each node is either a drug, target, disease, or adverse effect, and the edges define interactions between the drugs and targets, and adverse effect reactions to the drugs.

15. The non-transitory computer readable medium of claim 11, wherein the interactions between the drugs and targets are novel predicted and validated drug target interactions (DTIs), or predicted and validated DTIs, and wherein the adverse effect reactions to the drugs are either an inferred target-adverse drug reaction (ADR) association, or a clinically reported ADR.

16. The non-transitory computer readable medium of claim 11, wherein generating the deep learning network model comprises generating a drug vector matrix and a protein vector matrix by network embedding using the t-distributed stochastic neighbor embedding (t-SNE) algorithm.

17. The non-transitory computer readable medium of claim 11, wherein generating the deep learning network model comprises:
generating a random surfing model configured to capture graph structural information by generating a probabilistic co-occurrence (PCO) matrix for each of the plurality of networks of the biomedical network system;
generating a respective PPMI matrix based on each respective PCO matrix; and
creating a stacked denoising autoencoder to generate a low-dimensional vector representation of each vertex of a deep learning network model graph.

18. The non-transitory computer readable medium of claim 11, wherein the score prioritizer determines a score of new drug-target interactions (DTIs), wherein the prioritized score is determined by the matrix multiplication Sᵢⱼ=XᵢZYⱼ^{T}, wherein X is a matrix representation of drug features, Y is a matrix representation of biological target features, and Z is a positive unlabeled (PU) matrix of a known drug-biological target network that defines known relationships between drugs and biological targets.

19. A system to implement a deep learning network model, comprising:
a memory configured to store machine-readable instructions and data;
a processing unit configured to access the memory and execute the machine-readable instructions, the machine-readable instructions and data comprising:
a biomedical information library comprising information that includes a plurality of drugs, a plurality of biological targets, a plurality of diseases, and a plurality of adverse effects;
a biomedical network system comprising a plurality of networks covering chemical, genomic, phenotypic, and cellular profiles and which integrates biomedical data from one or more corporate entities;
a score prioritizer to determine new targets for the plurality of drugs based on a concatenation of a low-dimensional vector representation for each drug vertex in the biomedical network system, and a low-dimensional vector representation for each biological target vertex in the biomedical network system; and
a model generator configured to generate a deep learning network model that defines a plurality of relationships between the drugs, the biological targets, and the adverse effects from the plurality of networks;
wherein the plurality of relationships defined by the deep learning network model predicts target identification and repurposing for clinically failed drugs.

20. The system of claim 19, wherein the biomedical data from one or more corporate entities includes molecule-protein networks, drug-disease networks from clinical trials, and omics data including proteomics and RNA-seq.
